SEQUENCE LISTING (6 sequences)

SEQ ID NO: 1 - DNA, 939 bp, Homo sapiens
  misc_feature: Genbank/CR536554
atggcggctcagaagataaacgaggggctggaacacctcgccaaagcagagaaatacctgaaaactggttttttaaaatggaagccagattatgacagtgccgcttctgaatatggaaaagcagctgttgcttttaaaaatgccaaacagtttgagcaagcaaaagatgcctgcctgagggaagctgttgcccatgaaaataatagggctctttttcatgctgccaaagcttatgagcaagctggaatgatgttgaaggagatgcagaaactaccagaggccgttcagctaattgagaaggccagcatgatgtatctagaaaacggcaccccagacacagcagccatggctttggagcgagctggaaagcttatagaaaatgttgatccagagaaggctgtacagttatatcaacagacagctaatgtgtttgaaaatgaagaacgcttacgacaggcagttgaattactaggaaaagcctccagactactagtacgaggacgtaggtttgatgaggcggcactctctattcagaaagaaaaaaatatttataaggaaattgagaattatccaacttgttataagaaaacaattgctcaagtcttagttcatctacacagaaatgactatgtagctgcagaaagatgtgtccgggagagctatagcatccctgggttcaatggcagtgaagactgtgctgccctggaacagcttcttgaaggttatgaccagcaagaccaagatcaggtgtcagatgtctgcaactcaccgcttttcaagtacatggacaatgattatgctaagctgggcctgagtttggtggttccaggagggggaatcaagaagaaatcacctgcaacaccacaggccaagcctgatggtgtcactgccacggctgctgatgaagaggaagatgaatactcaggaggactatgctag

SEQ ID NO: 2 - protein, 312 aa, Homo sapiens
  misc_feature: Genbank/CAG38791
MAAQKINEGLEHLAKAEKYLKTGFLKWKPDYDSAASEYGKAAVAFKNAKQFEQAKDACLREAVAHENNRALFHAAKAYEQAGMMLKEMQKLPEAVQLIEKASMMYLENGTPDTAAMALERAGKLIENVDPEKAVQLYQQTANVFENEERLRQAVELLGKASRLLVRGRRFDEAALSIQKEKNIYKEIENYPTCYKKTIAQVLVHLHRNDYVAAERCVRESYSIPGFNGSEDCAALEQLLEGYDQQDQDQVSDVCNSPLFKYMDNDYAKLGLSLVVPGGGIKKKSPATPQAKPDGVTATAADEEEDEYSGGLC

SEQ ID NO: 3 - DNA, 3396 bp, Homo sapiens
  misc_feature: Genbank/NM_138571
cattaccaacgaggcgcaggggtcaggacgactctcggcagcgccattgcgcgccctctagtggcagccggttttgaggccggcctccggctttgaagttcctcaccgcgtctccttccctctccccaaagcctggatcaccgcccagcgtcaggcgaggggcgacgtctcgaggtaaaacggaggaggtgcgggacgcggagactgcgcgggcccggtagccctggagaggccgaggctctaggccgcgaggggcgggtgcaatggcggaggaacaggtgaaccgcagcgccggcctggcccccgactgtgaggcctcggcgactgcagaaactacggtttcctcagtggggacctgtgaagccgctggcaagtcaccagagcccaaggactacgacagcacctgcgtgttctgccggatcgcggggcggcaggacccgggcaccgaactcctgcactgcgagaatgaggacctaatttgcttcaaagatatcaaaccagcagcaactcatcattatcttgtggtgccaaagaagcatattggaaactgcagaactctaaggaaagatcaagtagaactggttgagaacatggtaactgttggaaaaaccattcttgaaagaaataatttcactgacttcacgaatgtgaggatgggttttcatatgccaccattctgttccatttcccacttgcaccttcatgttctggcaccagtggatcagcttggcttcttatccaagttggtttatagagtcaattcctattggtttatcacagctgatcacttgattgaaaaactaagaacatgaaaatgtcaagagtggaagatttttctaatcttggttcagcatgaagtggtatttaggtcccttttaagtctaattgcaattttaagatttgttgggttttatgagaggctgttacttagtggccttaaatcttttctgaatgtctgtttcctaagatctgtgatacagttatgtgaatattttgttactgacttgtttcaatggttacttgtataaggattttatatatatgatactatagataaaatcctatttaagacaaattctgttaatcaacaagggctctgtatttttttaagttaaaatattttcatttctcagtaagtagtcagttataatagtgatttatttatgaagaataaactactatagaaagtactttgtgggaacctttattactgtgaaggagaaaaatagctatttcttgacagttctatagttatacacataaattttcaaaataaagaataccatactacacaatgatctctgattttgctgagggaaggaattatatatgtgccactttttcctcaaatttcagatgccagttttcactttttgaagcattgcatctttttaataaatctatcctgaaagctatataatatatataatttatatcaatgaatttacaataactatcaaattggctatcagaattcactgtaatggaattttgcctctaataacataaagggggaagaaacataaagtggttctcttaataaaactagcagaaaatgttgctttatggcatgtttttgtatcaaagagaaatcagaggttaaactttgcttttatcatgaaaagggcctgatttgaaggtagagataggcttagatgagaaaaattgataacgcaatgttaatactattgatatttcataggtatattttgttagaaattggttttttggtgctaatctataggcacaatagtgaatatgaatgaagagtaggtgctgcaagttaatcctacattgtgccagtgtatattagtaccatgtaaatgaatatgaataaatgaacatgaggattaacgaactgttgaaaataggctgagatactagttttctttcttttttagagggggcatgatattaaagatagttttaagtaacaaattacttattacaaagttacaacatgtggaatcaatagttttatttgttcattaatttattcattcagcaaacatttgttgaacatttaatgtgtaccagatattgccaggatcaggaaataaagaactaaaaatggacattcagactgcaattgctagtacaggggtcaaacaaaatcagtgattagaatttagtgcgttaagttccctgatgccttcacagccagaagaggctgtgaagggataaacacttctgagagtgggtggtagtagaactgagtattcaagactgaatgttaggcaggtagacagtgactggttaggctgagaaacttacaagtattttcgttgagttctgcttccactattatttactttacaatggatatgaagttcagatttcatcttatttactgaaggtggagaaaggatgtggaagtaggggttatgggctctcaaaagtagatttagagagatttttttatcactgttttatgatatagttcactgagcacttacatagattaacagttacaagtttccataaatcagttagaatatgactagcttcagggaaggaattttcaacaactgcaatctttgattgttttactgtgggaacttgcagtgatataattgacaacattatttaacaataataggtaaagtaggccgggtgtggtggctcacgcctgtaatcctagcactttgggaggccgaggtgggtggatcacaaggtcaggagttcgagaccagcctggccaatatggtgaaaccccgtctcttctaaaaaaatacaaaaattagccgggtatggtggcgcatgcctgtagtcccagctactggggaggctgaggcaagagaattgcttgaactcgggaggcggaggtcgcagtgagccgagatcgtgccactgcactccagcctgggtgacagagtgagactttgtctcaaagaaaaaaaaacaataataggtaaagtatttaaggaattctcagatgccttatggacctcttcaaaaatgtggttagccaattgattcaacttttataggtttatatgctctcatgtaaacctatttgtttccgagatcagacgagatcgggcgcgttcagggtggtatggccgtagacaacctatttgttaaaaacccttggaaacacatcttttagtagaggttcttatttacttataaagattaataatcaaattactacaattaatatccatttagccttttcagtagtctaaatatttggtaactccctagccatctataacactagttcaaaacaaggctaattcataacctgtcttaaaaatgtttgaagtggtatcaagagcattgtttatgcatgttgtgatactatttgtggaactgtttaatgagctatttcaaactgctagaagaaaatgaaataaaaatggaaaaatactgtcaaaaaaaaaaaaaaaaaaa

SEQ ID NO: 4 - protein, 182 aa, Homo sapiens
  misc_feature: Genbank/NP_612638
MAEEQVNRSAGLAPDCEASATAETTVSSVGTCEAAGKSPEPKDYDSTCVFCRIAGRQDPGTELLHCENEDLICFKDIKPAATHHYLVVPKKHIGNCRTLRKDQVELVENMVTVGKTILERNNFTDFTNVRMGFHMPPFCSISHLHLHVLAPVDQLGFLSKLVYRVNSYWFITADHLIEKLRT

SEQ ID NO: 5 - DNA, 7509 bp, Homo sapiens
  misc_feature: Genbank/NM_173601
ggcggtgtccaggcttccgggcgggtagtccttcggctccggagccgcgactgcgctcgcctaggtggtgggcggggagggaaggaagggagcgggcgcaaggcggcggcggcggctgcgggtgaggaacttgttgcgcccgcggctgcgcggtgcccctccctccgctccagttcgtcggggcgggcgcggcggcggcggcgaaggaggagcgcggccggggcgatgcggcgctacctgcgcgtcgtggtgctgtgtgtggcctgcggcttctgctcgctcctttacgctttcagccagctcgccgtgtccctggaagaaggaacgggcggcggtggcgggaagccgcaggccgcggtggcttcctggctcgcgggcggcggacgcggcgccgtgagaggcgccggcgtcgcgggccccgcagcgcatcccggcgtgtcggacaggtgtaaagatttctctctgtgttactggaatccctattggatgctgccctctgatgtttgtggaatgaactgcttttgggaagcggcttttaggtacagtctgaaaatacagcctgttgagaaaatgcatctagctgtagttgcctgtggtgaaagactggaagaaactatgaccatgttgaagtcagctatcattttcagcatcaaacctcttcaattccatatttttgctgaagatcagctacatcatagctttaaaggcagacttgacaactggtcatttctacaaacatttaattatacgttataccccataacctttccaagtgagaatgcagcagagtggaaaaaactctttaaaccatgtgcttcgcagagattgttcttgccgttaatcctgaaagaagttgactcactattgtatgtcgacactgatatcctttttttacgaccagttgatgatatttggtctttactaaagaaatttaattccacacaaattgctgcaatggcaccagaacatgaggaacctcgaataggatggtataatcgctttgctaggcatccatattatggaaaaactggagtaaactctggagttatgttgatgaacatgactcgaatgagaaggaagtatttcaagaatgatatgacaactgtacgactacaatggggagatatacttatgccattgcttaaaaaatacaaactaaacatcacatggggtgatcaagatctattgaatatcgtgttttttcataatccagaaagcctttttgtttttccgtgtcaatggaattatcgaccagatcattgtatatatggaagcaattgccaagaagcagaagaaggaggaatctttattcttcatgggaacagaggtgtttaccatgacgataagcaaccagcatttagagctgtttatgaagcactgagaaattgttcttttgaagatgacaacatccgttccttattaaaacctttagaactggaactacaaaaaacagtgcatacatactgtggaaaaatttacaaaatatttatcaaacaactagcaaaaagtgtaagagatcgttatgccagatcaccaaaggaaaagtgattcttggtgactgcttaatcaaatggatgaaaacaaagaatcagaagataagtgtgaaggaatcgtcttggatgaagtattcaggaaggaattactcatctccagaataattttttttttcctaaagaagttaagtaagcagtattttcaggtaatgaagaataagttaaaatcttgggcctcaacattgaacattttttatctctgatgttttgtaatgttacttgctatcattccagtattgatgaaaatactattgaatgggtttaacctgcagacttctgttgactcatactctcaagagtggtaggggtgtgtagatggagaaaatgtacctcaaacagtgccaacactcaagactgtgagtagagcaataattttatgtcagcactaacctcactttaaaagtgtgagaaaaaagtttgtttacaggagcagaaacaggtctgttgtttctgaagaaatgtgatgtaactgatgtaaccattgacaatctatgtgtgcctttatacatttcatctctgttttaaaatatttttatgacaatcatgtttaaaattatttttagattacaagtaagctgcatgttaaaaattgagctgtgtaaggtagaggaaaaatagtgaaaactttgggattttaatctgtgtgtgcgtgtttgtgtgtgagagagagtgagaaatgtagtgtttcattttgtatgcattaaactgtcttgccaaaactcagatctaagattgttaaatagatatttggggaatttttttttaatcactttaaatgaaaaacgtcagctttactgggcattctggaagcaaaatatattatgctgatggtaaagtgagaaccttaagagtatactcatgatggtggaaagtgtgatggaccaaaatgcataagctatatacttcctgtgagtagtaattttagttacaatggagtagaaaaatatggtccatttaaacttgtcttctacctcataacggccttgcaagatacttttgaagaagcaaatgtctagagccttactttaagtaagttaaacaacttaagtggatgatttggaataagattgttacatccagctgtagaaaatgtggttttaattgggttgatgtgcatttttgaatatcacctaagacagttaatttttttaaatcttgagaaattacttctttggcattcttgacctgttagctaatataactatagattctgaatacttagcatatactatgctggggcacatatcttattttaccaacaatatgattatctatatggttatcttctttttctccctgatattttaatgctcaagtagaaaatggaaaatcatgaaggaaaaatacaacctgtgaaaattgtggcagttagctttgtgaagacacagtgacctctgtggagacatggaatatgcagaaagagatggttagtacagttgttctgtcgtctgcagttcagtcagggacaaattgagagaggaaagtttaagcagggtgcattagaagttgataaccagtccagcattcttagaacagatgggtttagagaacacgagttgtagttccttggcagaatgccatggtgaatataagaaactgtgaaattgagcttactgagtagaaaatgagaatactaaaaatgtgaagtttggagggtatgtattagatcatttctaatggtcctcaaataatgatcattgcctgcctgactatgtaagattcttgttgggagcttaagaatagattcctgagctaggcctttggagactctggcatgctgtgtctaggagggagtctgtaaaacaatttttaatgaagaaatttaaaaattacacaaaactagaatagtgcattggtactcaacatccagatctgacacttatcactatcttaccatgtttgcttcatttgtcatttttttgtttgcttaagtatcttaaaatcccatacattgtgttatttcactgctgtgaacactttgggtagataccacgatatcacacctaacagaggcagcagcagtccttcggcttcatctcatagccagtccttaagcaaatttccctgattatctcaaaaatcatcttctagttggtttgttagaatcaacagaatataagtcacatgatgatttcgtttttaacaatcacctcagatgactttgatgttagctactttgagaaccactggaaattattttgccaacttgaaagttcaagttaggataccacaattcttggcacgtttggtggattataaatgtgattttgaaatattagaggaagcatttggggttaatgacaggatatgaatttatattagtaagtttgataagataaaatttcctcagtgaacggagaatctcagccccatgggctctaattgattgggacttgtgtagagcgtggtcacagtttaatccaactggactgtatgctgtggtctcactcatgttgtggcttatttacactgtcacacacactacattgtggatcagttggaaggttcttcatctggtttatttagacctgacctaattgatcattttaccataaattgaattatagtgaaaactgtcttttgggtactgtttctacctaaaattaaaggtatcacaaaatatgtataataatttttaaatgaattttctgaatttatctttagacaaggtaattcttttgaattctaaagaaatgcagttcttggagttgatgagaggttaaattatagatggacaacttagagaacgtattccatcttcatataaatggtctttcataaaacatagtgtatctatgcagtttatataaactacatgatttattatttgagtctatgtattctgacctcagaactatctctgggctacagtattaccaaagtgggatgcctttaaaatgcagtcaaacttcatttgtatgaagtagaaaagaacttgggaaacctttaaaaatatatattgtgatttatgctttaagaaaatgcatacttttttctaatgaagagtagttcataatatataagggttttaaggccctgaaataacattagtgcttactcattgctctctcatcttgcctccagtcagatgttattggtatagtgaaaagaagaccagattttgaactagatctggattctctttttagtaactcaggcatgttatttacccttctctgtgccttaggtttttaagtacaatcagtgtacccttcaaacctcacaagatgaaactcagaagagataatggatttggaagcattttgcgtggcttatattatgccacgccagtgttactgttgaagttctgttttgttcgttgttttctgtacagagcgttcagtttactcaggctgaatgttttctgacatatatctccagtatgaatttgtttatgtaaggaagagtttttcaggaagagaaagtagagaagaaaagaatgatgtaactaaatcaaaaaagaaggaaaagagaggaagtgagaaaaggggtgaggaaagagaaaggagtaagaatggggatggcaaagggcagagtaagagaaaagatggaatgagtgtatggaaaattgagaagtagagatggagagaaatggtgtggaaacatttactttttgagttgtagccctcacactacatcagtttgcttgcctgggctctctggggtcaggaaaggtgctgacttccatggaatggggcagcagggcctgctgtatttggtttgttagtactagagttgtttcccaatcaccctactcactgatcatttagtgccaattcaacattttatgtcttttttttttagtggatcccagtttgtgagctaggacctgtggaaatacaagtatcttcctttttaactttattgttttagccatcccaagtgttagactgcaagactaactacatattccccagtggaggttggaagtggaggggactgcttcattaggtctgaagggggccaaggaatgtgttgcagcagttttcgcagatgatactgacgagaggccaggtttgggaatcactgccctgccttggatggcaaattaagcaataaattacacctatttattcctcagttgggatactttcagttgaaccattctgagtagatttaagagaacaaaagacagcagaaataccttaaaagatattattggctgtcttatttttcagatatttgtatttaataaataccttagtagtagtcagaacctctttgagatgtttttagaaagaccactgatttgttttttaaaataatttttttggttccttagagtagagaagaaaagtaggggtgcaatatgtcaggtgtggggccagttatccatttattaaatgcaatttcaagtgtcttaaaaacaataattgccttattgcattttaatatttaataaagtagattaaataaagatttgtattataacagttattcttttatccattgccaaagcaacccttttttgcctattaaaatctggctaaactgttctgatgttgtttcttaattcattaaacatgaaactaggttttacaaactaaattatataaagacagaagtctaaaaatggtaccagaaaatttttttttaaaaaattaatgtgttattcaaatatccagcaattaaagactcatgagaaaaacagaatagcatattagatgtaacagtttaaaatggattgcaaatcaatgtcagacttaaatagaaaatatttattatcacctcatttctaccaataatttcagatgtttaatgggactttgaaaagataggaaaaaatagaaatgaaaatttatctcacctttaatactgtgaatactatttggagggtccctggtcagacatttaaatatcaaacttaagaactgacatggagcctttgattagtttatatacaggatctgaatatttacacacatagcacactatagcaacctcttgtaaaaaatcatgaagatagaattcaacagtgtatattctattaaaaagtaaattggctgggtgtggtggctcatgcatgtaatcccagcactttgggaggccgaggcaggaggatcgcttgagcccaagagtttgagaccagcctaggtgacaaagtgagaccccatctctacaaaaaaattaaaaaattatccagatgtggtggtgtttgcctgcggtcctagctacacaggaagttgaggcaggaggattgcttgagtgcaagagtttgaggctgcagagagccaagattgcaccattgcactccagcctgggtgacagagcgagaccctgtctcaaaaaataaaggaaattggctttgactatgaaaaacagccattttcaacctttttataaaacctcttcagggaatattggaatattatttttgtcaggcaaaaataatccagggcttgagcattgctgtagaatatgttatctgttcaaaacttagcagagcacgcatgcatcacatgcattgtcatcagtatttgagagctgaaccaatggtttttgatggttgccgtaacagaagatctgtgaagaggatggtggtttgcacctgttcctgaagtcaaactggaaaaggttgcaggtatttcaattttctcaatcattttgcataaatgtattaatattttgagtagtaatagttctaaacctgcaggaaggcatcattgtatactgtggtatgtagagaatactactcttacactgctctttatcttaaaatgttttggtatactgtgaatattttcctctaagtatttggtgtattttgacagttattaaaggagaatataaaattaaagattctggtctaaactaagaataatggaaagttgcttcgtcctgccttagaggagttttcagttcatcctcttcttgagaaactatgtgttgctgttactaaataaacatttactcaataaacgttttatttcctcaaaaaaaaaaaaaaaaaaaaaaa

SEQ ID NO: 6 - protein, 440 aa, Homo sapiens
  misc_feature: Genbank/NP_775872
MRRYLRVVVLCVACGFCSLLYAFSQLAVSLEEGTGGGGGKPQAAVASWLAGGGRGAVRGAGVAGPAAHPGVSDRCKDFSLCYWNPYWMLPSDVCGMNCFWEAAFRYSLKIQPVEKMHLAVVACGERLEETMTMLKSAIIFSIKPLQFHIFAEDQLHHSFKGRLDNWSFLQTFNYTLYPITFPSENAAEWKKLFKPCASQRLFLPLILKEVDSLLYVDTDILFLRPVDDIWSLLKKFNSTQIAAMAPEHEEPRIGWYNRFARHPYYGKTGVNSGVMLMNMTRMRRKYFKNDMTTVRLQWGDILMPLLKKYKLNITWGDQDLLNIVFFHNPESLFVFPCQWNYRPDHCIYGSNCQEAEEGGIFILHGNRGVYHDDKQPAFRAVYEALRNCSFEDDNIRSLLKPLELELQKTVHTYCGKIYKIFIKQLAKSVRDRYARSPKEK

(19) United States
(12) Patent Application Publication
(10) Pub. No.: US 2019/0185851 A1
(43) Pub. Date: Jun. 20, 2019

(54) EXOSOME SECRETION INHIBITOR

(71) Applicant: THEORIA SCIENCE INC., Tokyo (JP)

(72) Inventors: Takahiro OCHIYA, Chuo-ku, Tokyo (JP); Yusuke YOSHIOKA, Adachi-ku, Tokyo (JP); Nobuyoshi KOSAKA, Machida-shi, Tokyo (JP)

(73) Assignee: THEORIA SCIENCE INC., Tokyo (JP)

(21) Appl. No.: 15/758,600

(22) PCT Filed: Aug. 30, 2016
(86) PCT No.: PCT/JP2016/075324

§ 371 Date: Mar. 8, 2018

(30) Foreign Application Priority Data
Sep. 9, 2015 (JP) 2015-177552

(51) Int. Cl. C12N 15/113; A61P 35/00; A61P 35/04

(52) U.S. Cl. CPC C12N 15/113; A61P 35/00; C12N 2310/141; C12N 15/1137; A61P 35/04

(57) ABSTRACT

An exosome secretion inhibitor containing a substance suppressing expression of NAPG, HINT3 or GXYLT1 gene, or a substance suppressing activity of NAPG, HINT3 or GXYLT1 protein. It is possible to suppress secretion of exosome, thereby making it possible to suppress proliferation or metastasis of cancer cells, so that the substance suppressing expression of the gene or activity of the protein can be used as an active ingredient of a pharmaceutical composition suitably used in treatment of cancer or suppression of cancer metastasis, and also the substance can provide a carcinostatic agent having a new action mechanism which specifically suppresses expression of the gene or activity of the protein.

### TECHNICAL FIELD

The present invention relates to an exosome secretion inhibitor. More specifically, the present invention relates to an exosome secretion inhibitor and a pharmaceutical composition, containing a substance suppressing expression of a particular gene or activity of a particular protein, and a method of screening a substance inhibiting exosome secretion, based on fluctuations of expression of a particular gene or activity of a particular protein.

### BACKGROUND ART

Most common features of cancer cells are rapid and uncontrollable proliferation as compared to normal cells. Therefore, much researches have been made on an approach in which a gene to be targeted by cancer therapy is found and proliferation of cancer cells are suppressed by suppressing expression or function of a target gene to treat cancer.

For example, Patent Publication 1 discloses miR-194 as a biomarker associated with the recurrence of liver cancer in a sample isolated from a patient with liver cancer. In addition, Patent Publication 2 describes that autoregulation loop associated with p53/HDM2 in multiple myeloma can be suppressed with miR-194.

In addition, it has been known that expression of NAPG gene is increased in endothelial cells of ovarian cancer (see, Patent Publication 3). It is reported that this NAPG gene is associated with a bipolar disorder or vesiculation in addition to the above disease (see, Non-Patent Publications 1 and 2).

On the other hand, it has been reported that exosome present in body fluid in a living body is secreted from various cells, for example, immunological cells or various cancer cells, and it has been remarked that exosome functions as a mediator of an intercellular communication in a living body and associated with physiological phenomenon and that the exosome is associated with diseases such as cancer.

For example, Non-Patent Publication 3 clarifies that exosome secreted by fibroblasts is involved in bosselation of lung cancer cells. In addition, it has been reported that exosome derived from melanoma promotes metastasis of primary focus, and that exosome which is secreted from metastatic focus by stimulation of neutral sphingomyelinase 2 (nSMase2) further promotes metastasis of cancers (see, Non-Patent Publications 4 and 5).

### PRIOR ART REFERENCES

### Patent Publications

Patent Publication 1: WO 2012/151212
Patent Publication 2: WO 2012/019053
Patent Publication 3: WO 2008/101118

### Non-Patent Publications

Non-Patent Publication 1: Neuroscience Letters, 457(2009), 159-162
Non-Patent Publication 2: Genes to Cells, (2005), 10, 989-999
Non-Patent Publication 3: Cell, 151, 1542-1556, Dec. 21, 2012
Non-Patent Publication 4: Nat Med., 2012 June; 18(6):883-891
Non-Patent Publication 5: J Biol. Chem., 2013, 288:10849-10859

### SUMMARY OF THE INVENTION

### Problems to be Solved by the Invention

While the relationship of each of a biomarker and cancer, and exosome and cancer is clarified, a biomarker regulating secretion of exosome has not yet been known.

An object of the present invention is to provide, as a new approach to cancer therapy, an exosome secretion inhibitor, a pharmaceutical composition, and a method of screening a substance for inhibiting exosome secretion, based on fluctuations of expression of a particular gene or activity of a particular protein by inhibiting secretion of exosome, thereby suppressing proliferation or metastasis of cancer cells to find genes which enable the treatment of cancer.

### Means to Solve the Problems

The present invention relates to the following [1] to [4]:
[1] An exosome secretion inhibitor containing a substance suppressing expression of NAPG, HINT3 or GXYLT1 gene, or a substance suppressing activity of NAPG, HINT3 or GXYLT1 protein.
[2] A pharmaceutical composition containing a substance suppressing expression of NAPG, HINT3 or GXYLT1 gene, or a substance suppressing activity of NAPG, HINT3 or GXYLT1 protein.
[3] An exosome secretion inhibitor containing miR-194.
[4] A method of screening a substance for inhibiting exosome secretion, including using expression of NAPG, HINT3 or GXYLT1 gene or activity of NAPG, HINT3 or GXYLT1 protein as an index to make a substance suppressing the expression or suppressing the activity a candidate compound.

### Effects of the Invention

According to the present invention, the exosome secretion can be suppressed by suppressing expression of NAPG, HINT3 or GXYLT1 gene, or suppressing activity of NAPG, HINT3 or GXYLT1 protein, which in turn makes it possible to suppress proliferation or metastasis of cancer cells. Therefore, the substance inhibiting expression of the above gene or activity of the above protein can be used as an active ingredient of a pharmaceutical composition suitably used in the treatment of cancer or suppression of cancer metastasis. The pharmaceutical composition of the present invention provides a carcinostatic agent having a new action mechanism which specifically suppresses expression of a particular gene or activity of the protein. Further, the present invention provides a method of screening an exosome secretion inhibitor which is effective as a substance for the treatment of cancer or a substance for suppression of metastasis using the above gene or protein as a target.

### BRIEF DESCRIPTION OF THE DRAWINGS

FIGS. 1A to D are diagrams showing the results of researching miRNA having an inhibitory action of exosome secretion. (A) is diagrams showing one example of Example 1, which show the results obtained by using siRNA against RAB27A (Applied Biosystems), RAB27B (Applied Biosystems), and nSMase2 (Applied Biosystems) as positive controls. Dotted line in the chart shows the amount of exosome secretion in a group added with negative control siRNA, and broken line shows the amount of exosome secretion in a group added with positive control siRNA against RAB27A.

FIG. 1(B) is graphs showing one example of the results of measurement of the amount of exosome secretion using an anti-CD63 antibody and an anti-CD9 antibody, which show a relative secretion amount to the secretion amount of negative control miRNA. Left graphs are the results obtained by using PC-3ML cells, and right graphs are the results obtained by using MDA-MB-231D3H2LN cells.

FIG. 1(C) is graphs showing one example of the results of comparing the number of exosome secretion particles per cell count. Left graph is the results obtained by using PC-3ML cells, and right graph is the results obtained by using MDA-MB-231D3H2LN cells.

FIG. 1(D) is graphs showing that the number of exosome secretion particles is changed by administration of LNA. Left graph is the results of the exosome secretion amount by carrying out LNA treatment using PC-3ML cells, and right graph shows an expression level of miR-194 itself.

FIGS. 2A to B are diagrams showing the results of identification of target genes of miR-194. (A) shows the results of analyzing and extracting genes which alter depending on the fluctuations of expression of miR-194 using a microarray.

FIG. 2(B) is a graph showing the results of analyzing and comparing an expression level of NAPG which is a candidate of a target gene using real-time PCR method. Left zone is the results obtained by using PC-3ML cells, and right zone is the results obtained by using MDA-MB-231D3H2LN cells.

FIGS. 3A to B are graphs showing the results of evaluations of inhibition of exosome secretion by siRNA. (A) is the results for MDA-MB-231D3H2LN cells, wherein the upper panel shows the results of measurement using an anti-CD9 antibody, and the lower panel shows the results of measurement using an anti-CD63 antibody, respectively. Dotted lines in the graphs show values of negative control (NC 1), and broken lines show values of positive control (RAB27A).

FIG. 3(B) is the results for PC-3ML cells, wherein the upper panel shows the results of measurement using an anti-CD9 antibody and the lower panel shows the results of measurement using an anti-CD63 antibody, respectively. Dotted lines in the graphs show values of negative control (Alistar), and broken lines show values of positive control (RAB27A).

FIG. 4 is graphs showing the results of evaluation of inhibition of exosome secretion by siRNA of NAPG. Left zone is the results for PC-3ML cells, and right zone is the results for MDA-MB-231D3H2LN cells.

FIG. 5 is a graph of the results of evaluation of inhibition of exosome secretion by siRNA using MDA-MB-231D3H2LN cells. The inhibition of exosome secretion was measured by using CD9 antibody.

FIG. 6 is graphs showing the results of evaluation of actions inhibiting cancer metastasis in an in vivo model. (A) is a graph of measurements of change in tumor sizes of primary focus by administration of siRNA, and (B) is a graph showing the number of micro metastasis to the lungs.

### MODES FOR CARRYING OUT THE INVENTION

The exosome secretion inhibitor of the present invention is greatly characterized in that the exosome secretion inhibitor contains a substance suppressing expression of NAPG gene, HINT3 gene or GXYLT1 gene, or a substance suppressing activity of NAPG protein, HINT3 protein or GXYLT1 protein. Incidentally, terms used herein are used in the meaning ordinarily used in the field, unless otherwise noted particularly.

It is known that particular biomarkers are involved in various cancers. On the other hand, it is known that an increase in the amount of exosome secretion is involved in the progress or metastasis of cancer. In view of the above, when the present inventors have studied remarking on biomarkers capable of regulating exosome secretion, it has been found that the exosome secretion can be suppressed by suppressing expression of NAPG gene, HINT3 gene or GXYLT1 gene, which in turn makes it possible to suppress the progress or metastasis of cancer.

The “NAPG gene” as used herein means a gene encoding human NAPG protein. A nucleotide sequence of human NAPG gene and an amino acid sequence of human NAPG protein are known, and for example, each is registered in GenBank and published as GenBank Accession ID: CR536554 as a nucleotide sequence of NAPG gene (SEQ ID NO: 1) and GenBank Accession ID: CAG38791 as an amino acid sequence of human NAPG protein (SEQ ID NO: 2). Conventionally, there have been only findings that expression level of NAPG gene is increased in endothelial cells in ovarian cancer and that NAPG gene is involved in bipolar disorder or vesiculation; however, the present inventors have found for the first time an action of suppressing exosome secretion by NAPG gene.

The “HINT3 gene” as used herein means a gene encoding human HINT3 protein. A nucleotide sequence of human HINT3 gene and an amino acid sequence of human HINT3 protein are known, and for example, each is registered in GenBank and published as GenBank Accession ID: NM_138571 as a nucleotide sequence of HINT3 gene (SEQ ID NO: 3) and GenBank Accession ID: NP_612638 as an amino acid sequence of human HINT3 protein (SEQ ID NO: 4). While it is reported that HINT3 gene functions as a hydrolase of phosphoramidic acid, the function thereof is not nearly known, and the present inventors have firstly found a suppression action of exosome secretion by HINT3 gene.

The “GXYLT1 gene” as used herein means a gene encoding human GXYLT1 protein. A nucleotide sequence of human GXYLT1 gene and an amino acid sequence of human GXYLT1 protein are known, and for example, each is registered in GenBank and published as GenBank Accession ID: NM_173601 as a nucleotide sequence of GXYLT1 gene (SEQ ID NO: 5) and GenBank Accession ID: NP_775872 as an amino acid sequence of human GXYLT1 protein (SEQ ID NO: 6). While it is reported that GXYLT1 gene functions as a polysaccharide synthase, a detailed analysis has not been carried out, and the present inventors have found for the first time an action of suppressing exosome secretion by GXYLT1 gene.

The human NAPG protein as used herein includes not only a protein consisting of the amino acid sequence of SEQ ID NO: 2, but also a mutant which may be generated in a human individual, including a protein in which one or more amino acids are deleted, substituted and/or added in the protein consisting of the sequence of SEQ ID NO: 2, generated by modifications based on polymorphism or mutation. However, these mutant human NAPG proteins have functions equivalent to those of the protein consisting of the amino acid sequence of SEQ ID NO: 2.

The “NAPG gene” as used herein includes not only a gene consisting of the nucleotide sequence of SEQ ID NO: 1, but also a mutant which may be generated in a human individual, including, for example, a gene in which one or more bases are deleted, substituted and/or added in the gene consisting of the nucleotide sequence of SEQ ID NO: 1, generated by modifications based on polymorphism or mutation. Further, the “NAPG gene” includes a mutant consisting of a nucleotide sequence having an identity of 80% or more, for example, 85% or more, 90% or more, 95% or more, 97% or more, 98% or more, 99% or more, 99.5% or more, 99.7% or more, or 99.9% or more of the nucleotide sequence of SEQ ID NO: 1. The identity of a nucleotide sequence can be determined by utilizing a known algorism such as BLAST or FASTA. Further, the “NAPG gene” includes a mutant consisting of a nucleotide sequence hybridizing to the gene consisting of the nucleotide sequence of SEQ ID NO: 1 under stringent conditions. Here, in the present specification, examples of stringent hybridization conditions include, for example, usually conditions of “1×SSC, 0.1% SDS at 37° C.” or so as washing conditions after hybridization. It is preferred that a complementary chain maintains a hybridized state with a positive chain to be subject even if the complementary chain is washed under the above conditions. More stringent hybridization conditions include, without being particularly limited to, washing conditions of “0.5×SSC, 0.1% SDS at 42° C.” or so, and further more stringent hybridization conditions include washing conditions of “0.1×SSC, 0.1% SDS at 65° C.” or so. In addition, the hybridization can be carried out in accordance with the methods described in Molecular Cloning: A Laboratory Manual, Second Edition (1989) (Cold Spring Harbor Laboratory Press), Current Protocols in Molecular Biology (1994) (Wiley-Interscience), DNA Cloning 1: Core Techniques, A Practical Approach, Second Edition (1995) (Oxford University Press), and the like. However, these mutant genes encode proteins having functions equivalent to those of the protein consisting of the amino acid sequence of SEQ ID NO: 2.

The human HINT3 protein used herein includes not only a protein consisting of the amino acid sequence of SEQ ID NO: 4, but also a mutant which may be generated in a human individual, including a protein in which one or more amino acids are deleted, substituted and/or added in the protein consisting of the sequence of SEQ ID NO: 4, generated by modifications based on polymorphism or mutation. However, these mutant human HINT3 proteins have functions equivalent to those of the protein consisting of the amino acid sequence of SEQ ID NO: 4.

The HINT3 gene as used herein includes not only a gene consisting of the nucleotide sequence of SEQ ID NO: 3, but also a mutant which may be generated in a human individual, including, for example, a gene in which one or more bases are deleted, substituted and/or added in the gene consisting of the nucleotide sequence of SEQ ID NO: 3, generated by modifications based on polymorphism or mutation. Further, the “HINT3 gene” includes a mutant consisting of a nucleotide sequence having an identity of 80% or more, for example, 85% or more, 90% or more, 95% or more, 97% or more, 98% or more, 99% or more, 99.5% or more, 99.7% or more or 99.9% or more of the nucleotide sequence of SEQ ID NO: 3. The identity of the nucleotide sequence can be determined in the same manner mentioned above. Further, the “HINT3 gene” includes a mutant consisting of a nucleotide sequence hybridizing to the gene consisting of the nucleotide sequence of SEQ ID NO: 3 under stringent conditions. However, these mutant genes encode proteins having functions equivalent to those of the protein consisting of the amino acid sequence of SEQ ID NO: 4.

The human GXYLT1 protein used herein includes not only a protein consisting of the amino acid sequence of SEQ ID NO: 6, but also a mutant which may be generated in a human individual, including proteins in which one or more amino acids are deleted, substituted and/or added in the protein consisting of the sequence of SEQ ID NO: 6, generated by modifications based on polymorphism or mutation. However, these mutant human GXYLT1 proteins have functions equivalent to those of the protein consisting of the amino acid sequence of SEQ ID NO: 6.

The “GXYLT1 gene” as used herein includes not only a gene consisting of the nucleotide sequence of SEQ ID NO: 5, but also a mutant which may be generated in a human individual, including, for example, a gene in which one or more bases are deleted, substituted and/or added in the gene consisting of the nucleotide sequence of SEQ ID NO: 5, generated by modifications based on polymorphism or mutation. Further, the “GXYLT1 gene” includes a mutant consisting of a nucleotide sequence having an identity of 80% or more, for example, 85% or more, 90% or more, 95% or more, 97% or more, 98% or more, 99% or more, 99.5% or more, 99.7% or more or 99.9% or more of the nucleotide sequence of SEQ ID NO: 5. The identity of the nucleotide sequence can be determined in the same manner mentioned above. Further, the “GXYLT1 gene” includes a mutant consisting of a nucleotide sequence hybridizing to the gene consisting of the nucleotide sequence of SEQ ID NO: 5 under stringent conditions. However, these mutant genes encode proteins having functions equivalent to those of the protein consisting of the amino acid sequence of SEQ ID NO: 6.

The “substance suppressing expression of gene” as used herein is not particularly limited, so long as the substance is a substance suppressing transcription of mRNA of a target gene, a substance degrading mRNA transcribed, or a substance suppressing translation of a protein from mRNA. The substance includes a nucleic acid, and examples include siRNA, miRNA, an antisense oligonucleotide and a ribozyme, and an expression vector thereof. Among them, siRNA, miRNA, an antisense oligonucleotide, and an expression vector thereof are preferred, and siRNA and an antisense oligonucleotide are more preferred. The “substance suppressing expression of gene” includes a protein, a peptide, or other low-molecular compounds in addition to the above. Here, the target gene in the present invention is NAPG gene, HINT3 gene, or GXYLT1 gene.

The “siRNA” as used herein is an RNA molecule having a double stranded RNA moiety composed of from about 15 to 40 bases, in which the siRNA has a function of cleaving mRNA of a target gene having a sequence complementary to an antisense strand of the above siRNA to suppress expression of the target gene. In particular, the siRNA in the present invention is an RNA containing a double stranded RNA moiety consisting of a sense RNA strand consisting of a sequence homologous to continuous RNA sequence in mRNA of NAPG gene, HINT3 gene, or GXYLT1 gene, and an antisense RNA strand consisting of a sequence complementary to the sense RNA sequence. The design and production of the siRNA and mutant siRNA described later are within the scope of skill of one of ordinary skill in the art.

The “miRNA” as used herein refers to a RNA molecule having a double stranded RNA moiety composed of from about 21 to 25 bases, which has a function of suppressing expression of a target gene by binding to mRNA of a target gene. In particular, miRNA in the present invention is an RNA containing a double stranded RNA moiety composed of a sense RNA strand consisting of a sequence homologous to continuous RNA sequences in mRNA of NAPG gene, HINT3 gene, or GXYLT1 gene, and an antisense RNA strand consisting of a sequence complementary to the sense RNA sequence. The design and production of the miRNA and mutant siRNA set forth below are within the scope of skill of one of ordinary skill in the art.

The length of the double stranded RNA moiety of the siRNA is preferably from about 15 to 40 bases, more preferably from 15 to 30 bases, even more preferably from 15 to 25 bases, even more preferably from 18 to 23 bases, and still even more preferably from 19 to 21 bases as a base. In addition, the length of the double stranded RNA moiety of the miRNA is preferably from about 21 to 25 bases, and more preferably from 22 to 24 bases as a base. The end structures of a sense strand or an antisense strand of the siRNA and the miRNA are not particularly limited, and can be appropriately selected depending upon the purpose. For example, the end structure may be blunt ended or may have a protrusion end (overhang), and one having a 3′ end protrusion is preferred. The siRNA and miRNA having an overhang composed of several bases, preferably from 1 to 3 bases, and more preferably 2 bases at a 3′ end of the sense RNA strand and the antisense RNA strand are preferred because they are highly likely to have large effects of suppressing expression of a target gene. The kinds of bases of overhang are not particularly limited, and may be any one of bases constituting an RNA or bases constituting a DNA.

Further, the siRNA and the miRNA in which one or more nucleotides are deleted, substituted, inserted and/or added in one or both of the sense strand or the antisense strand of the above siRNA and miRNA can be also used in the exosome secretion inhibitor of the present invention. Here, the one or more bases are not particularly limited, and are preferably from 1 to 4 bases, more preferably from 1 to 3 bases, and even more preferably 1 or 2 bases. Concrete examples of the mutations include a mutation in which the number of bases of the overhang moiety at a 3′ end is from 0 to 3; a mutation in which a nucleotide sequence of the overhang moiety at a 3′ end is changed to other nucleotide sequences; a mutation in which the length of the above sense RNA strand is different from the length of the above antisense RNA strand by from 1 to 3 bases by having insertion, addition or deletion of bases; a mutation in which a base is substituted with another base in the sense strand and/or the antisense strand, and the like, but not limited thereto. However, it is required that the sense strand and the antisense strand can be hybridized in these mutants siRNA and miRNA, and that these mutants siRNA and miRNA have ability of suppressing gene expression equivalent to those of siRNA and miRNA without mutations.

Further, the siRNA and the miRNA may be a molecule in which either one end is closed, for example, siRNA and miRNA having a hairpin structure, a short hairpin RNA (shRNA). The shRNA is an RNA containing a sense strand RNA of a particular sequence of a target gene, an antisense strand RNA consisting of a sequence complementary to the sense strand RNA, and a linker sequence linking both the strands, in which the sense strand moiety and the antisense strand moiety are hybridized to form a double stranded RNA moiety.

It is desired that the siRNA and the miRNA do not exhibit so-called off-target effects during clinical use. The off-target effects refer to an action of suppressing expression of another gene partially having homology to the siRNA and the miRNA used other than the target gene. In order to avoid the off-target effects, it is possible to previously confirm that the candidates siRNA and miRNA do not have a cross-reaction by utilizing a DNA microarray or the like. In addition, the off-target effects can be avoided by confirming whether or not there are any genes containing parts having high homologies to the sequences of the candidates siRNA and miRNA other than the gene to be targeted, using known database provided by NCBI (National Center for Biotechnology Information) or the like.

In order to produce the siRNA and the miRNA of the present invention, known methods such as a method according to a chemical synthesis and a method using gene recombinant technology can be properly used. In the method according to synthesis, a double stranded RNA can be synthesized according to the ordinary method on the basis of the sequence information. In addition, in the method using a gene recombinant technology, an expression vector into which a sense strand sequence and an antisense strand sequence are incorporated is constructed, and the vector is introduced into a host cell, whereby each of a sense strand RNA and antisense strand RNA produced by transcription can then be generated by acquirement. In addition, a desired double stranded RNA can be produced by expressing shRNA forming a hairpin structure, which contains a sense strand RNA of a particular sequence of a target gene, an antisense strand RNA consisting of a sequence complementary to the sense strand RNA and a linker sequence linking both the strands.

The siRNA and the miRNA may have a DNA in a part of nucleic acids constituting the siRNA and miRNA, so long as the siRNA and the miRNA have activity of suppressing expression of a target gene. In addition, the siRNA and the miRNA may be a nucleic acid in which the entire or a part of nucleic acids constituting the siRNA and the miRNA is modified, so long as the siRNA and the miRNA have activity of suppressing expression of a target gene.

The modified nucleic acid means a nucleic acid having a structure different from a naturally occurring nucleic acid because of modification provided in nucleoside (base site or sugar site) and/or internucleoside linkage site. The “modified nucleoside” constituting the modified nucleic acids includes, for example, abasic nucleoside; arabinonucleoside, 2′-deoxyuridine, α-deoxyribonucleoside, β-L-deoxyribonucleoside, other nucleosides with sugar modification; peptide nucleic acid (PNA), phosphate group-bound peptide nucleic acid (PHONA), locked nucleic acid (LNA), morpholino nucleic acid, and the like. The above nucleoside with sugar modification includes a nucleoside with a sugar modification such as substituted pentose such as 2′-O-methylribose, 2′-deoxy-2′-fluororibose, 3′-O-methylribose; 1′,2′-deoxyribose; arabinose; substituted arabinoses; and hexose and alpha-anomer. These nucleosides may be a modified base with modified at a basic site. The modified base includes, for example, pyrimidines such as 5-hydroxycytosine, 5-fluorouracil, and 4-thiouracil; purines such as 6-methyladenine and 6-thioguanosine; and other heterocyclic base, and the like.

The “modified internucleoside linkage” constituting the modified nucleic acid includes, for example, an alkyl linker, a glyceryl linker, an amino linker, a poly(ethylene glycol) linkage, a methyl phosphonate internucleoside linkage; a non-natural internucleoside linkage such as a methyl phosphonothioate, a phosphotriester, a phosphothiotriester, a phosphorothioate, a phosphorodithioate, a triester prodrug, a sulfonate, a sulfonamide, a sulfamate, formacetal, N-methyl hydroxylamine, a carbonate, a carbamate, a morpholino, a boranophosphonate, and a phosphoramidate.

An oligonucleotide complementary to the mRNA of a target gene is referred to as an “antisense oligonucleotide,” and the antisense oligonucleotide forms a double strand with a gene to be targeted (mRNA), thereby suppressing an action of mRNA. The “antisense oligonucleotide” includes not only those completely complementary to a gene to be targeted (mRNA), and may have a few mismatches, so long as the antisense oligonucleotide can stably hybridize to mRNA.

The antisense oligonucleotide may be modified. The antisense oligonucleotide is less likely to be degraded in a living body by providing proper modification, whereby expression of a target gene can be more stably inhibited. The modified oligonucleotides described above include a modified antisense oligonucleotide such as an S-oligo form (phosphorothioate form), a C-5 thiazole form, a D-oligo form (phosphodiester form), an M-oligo form (methyl phosphonate form), a peptide nucleic acid form, a phosphodiester linkage form, a C-5 propynyl pyrimidine form, a 2-O-propylribose, and a 2′-methoxyethoxyribose form. Further, the antisense oligonucleotide may be substituted or modified with a sulfur atom in at least a part of oxygen atoms constituting a phosphate group. The antisense oligonucleotide described above is particularly excellent in nuclease resistance and affinity to RNA. The antisense oligonucleotide substituted or modified with a sulfur atom in at least a part of oxygen atoms constituting a phosphate group includes, for example, an oligonucleotide such as an S-oligo form.

The antisense oligonucleotide (or derivative thereof) can be synthesized by an ordinary method, and, for example, can be easily synthesized with a commercially available DNA synthesizer (for example, a synthesizer manufactured by Applied Biosystems, or the like). A synthesizing method includes a solid phase synthesizing method using a phosphoramidite, a solid phase synthesizing method using a hydrogen phosphonate, and the like.

While the “ribozyme” refers to an RNA having an enzymatic activity for cleaving a nucleic acid, recently it has been clarified that an oligo DNA having a nucleotide sequence of the enzymatic activity site also has activity of cleaving nucleic acids. Therefore, in the present specification, the ribozyme is used as a concept also encompassing a DNA, so long as a DNA has a sequence-specific nucleic acid cleavage activity. Concretely, the ribozyme is capable of specifically cleaving mRNA or initial transcripts encoding a target gene inside the coding region (including intron portions in a case of initial transcripts). Most useful ribozyme includes a self-splicing RNA found in an infective RNA such as viroid or virusoid, and a hammerhead form, a hairpin form or the like is known. The hammerhead form exhibits enzymatic activity in a size of about 40 bases or so, and only the target mRNA can be specifically cleaved by making several bases at both ends adjoining a portion taking a hammerhead structure (about 10 bases or so in total) into a sequence complementary to a desired cleavage site of mRNA. Further, when the ribozyme is used in the form of an expression vector containing a DNA encoding the ribozyme, in order to promote transition of the transcription product into a cytoplasm, the ribozyme can also be made into a hybrid ribozyme in which a sequence with a modified tRNA is further linked (Nucleic Acids Res., 29(13): 2780-2788 (2001)).

The substance suppressing expression of a target gene may be a nucleic acid molecule such as siRNA, miRNA, an antisense oligonucleotide or a ribozyme, and an expression vector encoding the nucleic acid molecule. In the expression vector, an oligonucleotide or a polynucleotide encoding the above nucleic acid molecule must be operably linked to a promoter capable of exhibiting a promoter activity in cells of a mammal to be administered. The promoter used is not particularly limited, so long as the promoter is capable of functioning in the mammal to be administered, and the promoter includes, for example, a pol III promoter (for example, a tRNA promoter, a U6 promoter, an H1 promoter), a promoter for mammal (for example, a CMV promoter, a CAG promoter, an SV40 promoter), and the like.

The expression vector preferably contains a transcription termination signal, i.e. a terminator region, downstream of an oligo(poly)nucleotide encoding the nucleic acid molecule. Further, the expression vector can additionally contain a selection marker gene for selection of transformed cells (a gene giving resistance against an agent such as tetracycline, ampicillin, kanamycin, hygromycin, or phosphinothricin, a gene complementing auxotrophic mutation, and the like).

A backbone vector used as the expression vector is not particularly limited, and includes, for example, a plasmid vector and a virus vector. A vector suitable for administration to a mammal such as a human includes a virus vectors such as retrovirus, adenovirus, adeno-associated virus, herpes virus, vaccinia virus, pox virus, polio virus, Sindbis virus, and Sendai virus.

The expression vector of the present invention includes an expression vector of siRNA, miRNA, an antisense oligonucleotide or a ribozyme, and among them, an expression vector of siRNA is preferred.

These “substances suppressing expression of genes” have some effects of inhibiting an exosome secretion, so that proliferation or metastasis of cancer cells in broad ranges including lung cancer, colorectal cancer, pancreatic cancer, breast cancer and the like is suppressed, or the cancer cells are diminished, whereby the substances can be used as a pharmaceutical composition.

The “substance suppressing activity of protein” as used herein is not particularly limited, so long as the substance is a substance suppressing activity of a target protein. Examples of the substance include a protein, a peptide, an antibody, other low-molecular compounds or the like. Among them, a peptide, an antibody and low-molecular compounds are preferred, and an antibody and low-molecular compounds are more preferred. Here, in the present invention, the target protein is NAPG protein, HINT3 protein or GXYLT1 protein.

In the present invention, the substance suppressing expression of NAPG, HINT3 or GXYLT1 gene or the substance suppressing activity of NAPG, HINT3 or GXYLT1 protein can be used as an active ingredient of a pharmaceutical composition. The above pharmaceutical composition can be used as a pharmaceutical composition for treating cancer or a pharmaceutical composition for suppressing cancer metastasis by administering the composition to a living body.

Accordingly, the present invention also provides a pharmaceutical composition containing a substance suppressing expression of NAPG, HINT3 or GXYLT1 gene or a substance suppressing activity of NAPG, HINT3 or GXYLT1 protein as an active ingredient.

When the pharmaceutical composition of the present invention is used, for example, for cancer as a subject to be treated, the kinds of cancer are not particularly limited. In addition, the cancer may be solid cancer or invasive cancer. Concretely, the cancer includes, for example, urinary bladder cancer, breast cancer, colon cancer, colorectal cancer, renal cancer, liver cancer, lung cancer, small cell lung cancer, esophageal cancer, gallbladder cancer, ovarian cancer, pancreatic cancer, gastric cancer, cervical cancer, thyroid cancer, prostate cancer, squamous cancer, skin cancer, bone cancer, lymphoma, leukemia and brain tumor. Especially, the application of the pharmaceutical composition of the present invention is expected for breast cancer and colorectal cancer.

In addition, the pharmaceutical composition of the present invention can be used not only for treatment of cancer, but also for prevention of recurrence of cancer after treatment and suppression of metastasis. Accordingly, a preferred embodiment of the pharmaceutical composition of the present invention includes a pharmaceutical composition for treatment of cancer, a pharmaceutical composition for prevention of recurrence of cancer, and a pharmaceutical composition for suppression of cancer metastasis.

As the pharmaceutical composition of the present invention, both dosage forms of an oral administration and a parenteral administration can be adopted. In a case of a parenteral administration, the pharmaceutical composition can also be directly administered to a tumor site.

The pharmaceutical composition of the present invention can be produced into a formulation in accordance with a conventional method, and the pharmaceutical composition may contain a pharmaceutically acceptable carrier and an additive. The carrier and the additives as mentioned above include water, a pharmaceutically acceptable organic solvent, a collagen, a polyvinyl alcohol, a polyvinyl pyrrolidone, a carboxyvinyl polymer, sodium carboxymethyl cellulose, a sodium polyacrylate, sodium alginate, a water-soluble dextran, sodium carboxymethyl starch, pectin, methyl cellulose, ethyl cellulose, xanthan gum, gum arabic, casein, agar, a polyethylene glycol, diglycerol, glycerol, propylene glycol, Vaseline, a paraffin, stearyl alcohol, stearic acid, human serum albumin, mannitol, sorbitol, lactose, a surfactant accepted as a pharmaceutical additive, and the like.

The additives are selected alone or in a proper combination of two or more kinds among the above list depending upon the dosage forms of the pharmaceutical composition of the present invention. The dosage form can be administered as a tablet, a capsule, a fine powder, a powder, a granule, a solution, a syrup, or in a suitable dosage form in a case of an oral administration. In a case of parenteral administration, the dosage form includes a transpulmonary dosage form (for example, a form delivered by using a nebulizer or the like), a transnasal administration dosage form, a transcutaneous administration dosage form (for example, an ointment or a cream), an injectable dosage form and the like. In a case of an injectable dosage form, the dosage form can be systemically or locally administered by an intravenous injection, an intramuscular injection, an intraperitoneal injection, a subcutaneous injection or the like.

In a case where the substance suppressing expression of a target gene is a nucleic acid molecule such as siRNA, miRNA, an antisense oligonucleotide or a ribozyme, or an expression vector encoding the nucleic acid, it is possible to introduce the substance suppressing expression into a phospholipid vesicle such as a liposome to make the vesicle into a pharmaceutical composition of the present invention.

The dose of the pharmaceutical composition of the present invention varies depending upon age, sex, symptom, administration route, the number of administration or dosage form. A method of administration is properly selected depending upon age or symptom of a patient. An effective dose is preferably 0.01 μg to 1,000 mg, and more preferably 0.1 μg to 100 μg per administration per 1 kg of body weight. However, the above therapeutic agent is not limited to these doses.

In a further embodiment, the present invention provides:

(i) a method of treating cancer including administering a substance suppressing expression of NAPG, HINT3 or GXYLT1 gene, or a substance suppressing activity of NAPG, HINT3 or GXYLT1 protein;
(ii) a substance suppressing expression of NAPG, HINT3 or GXYLT1 gene, or a substance suppressing activity of NAPG, HINT3 or GXYLT1 protein which is usable in treatment of cancer; and
(iii) use of a substance suppressing expression of NAPG, HINT3 or GXYLT1 gene, or a substance suppressing activity of NAPG, HINT3 or GXYLT1 protein in the preparation of a therapeutic agent of cancer.

The above “substance suppressing expression of gene” or “substance suppressing activity of protein” is as explained above. In addition, in a case where siRNA is used as “a substance suppressing expression of gene,” a preferred siRNA is as mentioned above.

In another embodiment, the present invention provide a method of screening an exosome secretion inhibitor, including using an exosome amount as an index to have a substance suppressing secretion of exosome as a candidate compound.

A substance to be tested subjected to the method of screening may be any known compounds and novel compounds, and the substance includes, for example, a nucleic acid, a saccharide, a lipid, a protein, a peptide, an organic low-molecular compound, a compound library produced using a combinatorial chemistry technique, a random peptide library produced in accordance with a solid phase synthesis or a phage display method, a naturally occurring component derived from microbes, animals, plants, marine organisms etc., and the like.

The method of screening the present invention preferably includes methods of the following embodiment 1 and embodiment 2. The screening method of the embodiment 1 includes the following steps (a1) to (c1):

(a1) contacting a cell of which exosome amount is measurable with a substance to be tested;
(b1) measuring the exosome amount in the cell contacted with the substance to be tested to compare the measurement values with an exosome amount in a control cell without contacting the substance to be tested; and
(c1) selecting the substance to be tested as a substance for inhibiting exosome secretion in a case where the exosome amount in the cell contacted with the substance to be tested is lowered than the exosome amount in the control cell without contacting the substance to be tested.

The method of the embodiment 2 includes the following steps (a2) to (c2):

(a2) contacting a cell of which exosome amount is measurable with a substance to be tested;
(b2) measuring the exosome amount in the cell contacted with the substance to be tested to compare the measurement values with the exosome amount previously measured before contacting the cell with the substance to be tested; and
(c2) selecting the substance to be tested as a substance for inhibiting exosome secretion in a case where the exosome amount in the cell contacted with the substance to be tested is lowered more than the exosome amount in the cell before contacting with the substance to be tested.

In the steps (a1) and (a2), the cell of which exosome amount is measurable and the substance to be tested are placed under contact conditions. The contact of the cell with the substance to be tested is carried out in a culture medium.

The cell of which exosome amount is measurable includes nearly all animal cells, and the examples thereof include a disease cell such as a cancer cell, and a normal cell such as a vascular endothelial cell, an epidermal cell, a nerve cell, and an immunological cell. Here, as these cells, an animal cell, for example, a mammalian cell of mouse, rat, hamster, guinea pig, rabbit, dog, monkey or human can be used, among which a cell derived from human is desired.

The cell culture medium in which a cell of which exosome amount is measurable is contacted with a substance to be tested is properly selected depending upon the kinds of cells used and the like, and the medium is, for example, minimum essential medium (MEM) containing from about 5 to about 20% of fetal bovine serum, Dulbecco's modified minimum essential medium (DMEM), RPMI1640 medium, 199 medium or the like. The culture conditions are also properly determined depending upon the kinds of cells usable and the like, and a pH of the medium is from about 6 to about 8, a culture temperature is usually from about 30° to about 40° C., and a culture time is from about 12 to about 144 hours.

In the step (b1), the exosome amounts of the cell contacted with the substance to be tested and the control cell without contacting the substance to be tested are measured, and in the step (b2), the exosome amounts of the cell contacted with the substance to be tested before and after the contact are measured. The measurement of exosome amounts can be carried out in accordance with a known method, and the amount can be calculated as an exosome amount per cell. Concretely, preferred examples include, for example, a mass spectrometry and a method using an antibody which is capable of specifically recognizing a marker to be measured. Here, the exosome amount in the control cell without contacting can be newly measured every time the exosome amount in the cell contacted with substance to be tested is measured, but a previously measured exosome amount can be also utilized. The measurement of the cell count can be carried out in accordance with a known method.

The mass spectrometry includes mass spectrometry using MALDI (matrix-assisted laser desorption ionization) type mass spectrometer, ESI (electrospray ionization) type mass spectrometer and the like, and the method may be a method of quantification from a partial peptide of a protein. Among them, MALDI-TOF MS using MALDI type spectrometer is preferred.

The method using an antibody, which is an immunoassay, includes, for example, Western blotting, radioimmunoassay (RIA), enzyme-linked immunosorbent assay (ELISA or EIA), luminescent immunoassay, fluorescent immunoassay (exoscreen or the like). As the antibodies used in the method, since antigens which are specifically expressed on a membrane surface of exosome (for example, CD9, CD63, CD147, EPCAM or the like) are known, antibodies capable of specifically binding to these antigens can be used (for example, WO 2013/099925). The antibody as described above can be produced in accordance with a method which is well known to one of ordinary skill in the art, and for example, a monoclonal antibody or a fragment thereof can be suitably used. In addition, the above monoclonal antibody or the fragment thereof can be labeled or formed into a solid phase in accordance with an ordinarily known method and used. Here, “a fragment of monoclonal antibody” as used herein means a part of the above-mentioned monoclonal antibody, the fragment having specific bindability against a target protein in the same manner as the above monoclonal antibody. Concrete examples include a Fab, a F(ab′)₂, a Fab′, a single chained antibody (scFv), a disulfide-stabilized antibody (dsFv), a dimerized V region fragment (Diabody), a peptide containing a CDR, and the like.

In the step (c1), the exosome amount in the cell contacted with the substance to be tested is compared to the exosome amount in the control cell without contacting the substance to be tested, and in the step (c2), the exosome amounts before and after contacting with the substance to be tested are compared. The comparison of the exosome amounts is preferably carried out based on the presence or absence of significant differences. For example, in a case where the exosome amount in the cell contacted with the substance to be tested is lowered than the exosome amount in the control cell without contacting the substance to be tested, or the exosome amount in the cell after contacting the substance to be tested is lowered than the exosome amount in the cell before contacting with the substance to be tested in a statistically significant amount, preferably by two times or more, more preferably three times or more, even more preferably four times or more, and even more preferably five times or more, it is preferable to select the substance to be tested as a substance for inhibiting exosome secretion. As a test method in statistical processing, a known test method in which the presence or absence of significance can be judged may be properly used, and are not particularly limited thereto. For example, Student t test and multiple comparison test can be used.

In addition, in another embodiment, the present invention provides a method of screening a therapeutic agent for cancer, including using an exosome amount as an index to have a substance suppressing secretion of exosome as a candidate compound. The concretely used sample and method are same as in the method of screening an exosome secreting inhibitor of the present invention.

### EXAMPLES

The present invention will be described based on Examples, which are exemplifications for more fully understanding the present invention, without intending to limit the scope of the present invention to these Examples. Here, “ordinary temperature” used in the present Examples means from 15° to 30° C.

### Example 1 (Search of miRNA Having Inhibitory Action of Exosome Secretion)

The amount 1×10⁴ cells of PC-3ML cells (Xenogen) or MDA-MB-231D3H2LN cells (Xenogen) were seeded to each well in a serum-containing medium in a 96-well plate. Next day, the culture supernatant was exchanged with advanced RPMI (aRPMI; serum free, antibiotics free) (90 μL/well).

To each well of a separately furnished 96-well plate (PCR plate), 5 μL of 2 μM miRNA and 10 μL of aRPMI were mixed, for each miRNA in a micro RNA library (Accu Target Human Library mimic).

Next, DharmaFECT1 Transfection Reagent (Thermo Scientific) was diluted with aRPMI so as to have a concentration of 10 μL/well, and the solution was incubated at an ordinary temperature for 5 minutes, and aRPMI was added thereto so as to have a concentration of 90 μL/well. The solution was added in an amount of 90 μL per well of a PCR plate to which the above miRNA was injected, and the mixture was incubated at an ordinary temperature for 30 minutes. After the incubation at an ordinary temperature for 30 minutes, 90 μL of the incubated mixture was added to each well of a 96-well plate in which PC-3ML cells or MDA-MB-231D3H2LN cells were seeded. Next day, the culture supernatant was removed, and new aRPMI was added thereto in an amount of 60 μL. After additional 24 hours, 10 μL of the culture supernatant was used for quantification of the amount of exosome secretion, and the remaining supernatant was used for quantification of the cell count. Here, each quantification was carried out in accordance with the following methods.

<Quantification of Exosome Amount>

The collected culture supernatant is added to each well of a 96-well white plate in an amount of 10 μL each.

Biotinylated anti-CD9 antibodies (SHIONOGI & Co., Ltd., Clone 12A12) and biotinylated anti-CD63 antibodies (SHIONOGI & Co., Ltd., Clone 8A12) are diluted with AlphaLISA Universal Buffer (PerkinElmer) to a concentration of 5 nM. On the other hand, anti-CD9 antibodies (SHIONOGI & Co., Ltd., Clone 12A12) and anti-CD63 antibodies (SHIONOGI & Co., Ltd., Clone 8A12), each is conjugated with AlphaLISA Acceptor Beads, are diluted with AlphaLISA Universal Buffer (PerkinElmer) to a concentration of 50 μg/mL.

To the culture supernatant are added 10 μL of each of 5 nM biotinylated antibodies and 50 μg/mL antibodies bound to AlphaLISA Acceptor Beads, and the mixture is incubated at 37° C. for 1 hour under shading conditions.

The amount 5 mg/mL AlphaScreen streptavidin donor beads (PerkinElmer) is diluted 62.5 times with AlphaLISA Universal Buffer, the diluted solution is added to each well in an amount of 25 μL, TopSeal-A is adhered to the plate and incubated at 37° C. for 30 minutes under shading conditions, and the fluorescent intensity is measured with Enspire.

<Quantification of Cell Count>

To the collected culture supernatant is added an MTS solution (Dojindo, Cell Counting Kit-8) so as to have a concentration of 50 μL/well, and the mixture is incubated at 37° C. for 1 hour to measure an increase in cell count.

The exosome amount obtained was divided by the cell count to calculate an exosome secretion amount per cell count, to compare the effects of 2,042 kinds of miRNAs in a micro RNA library.

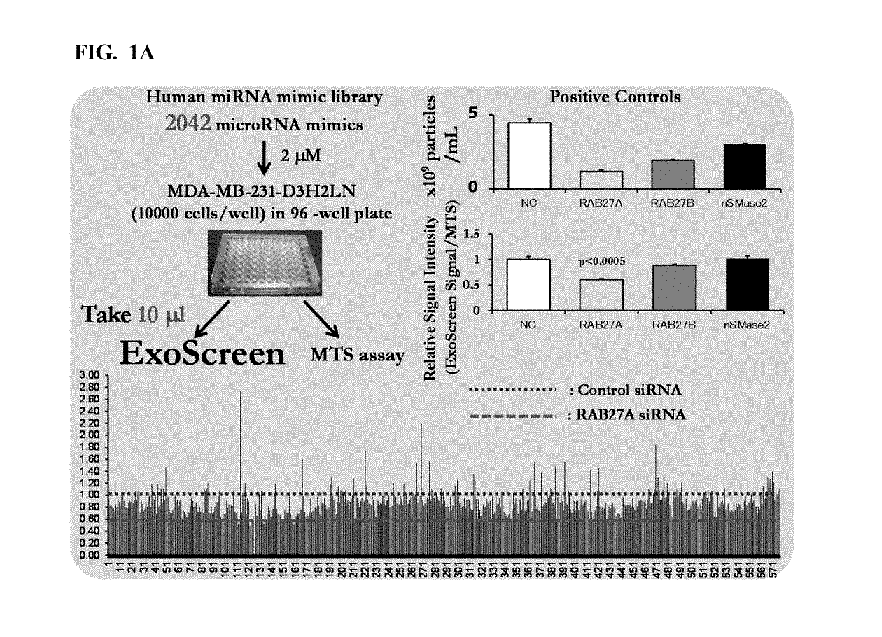

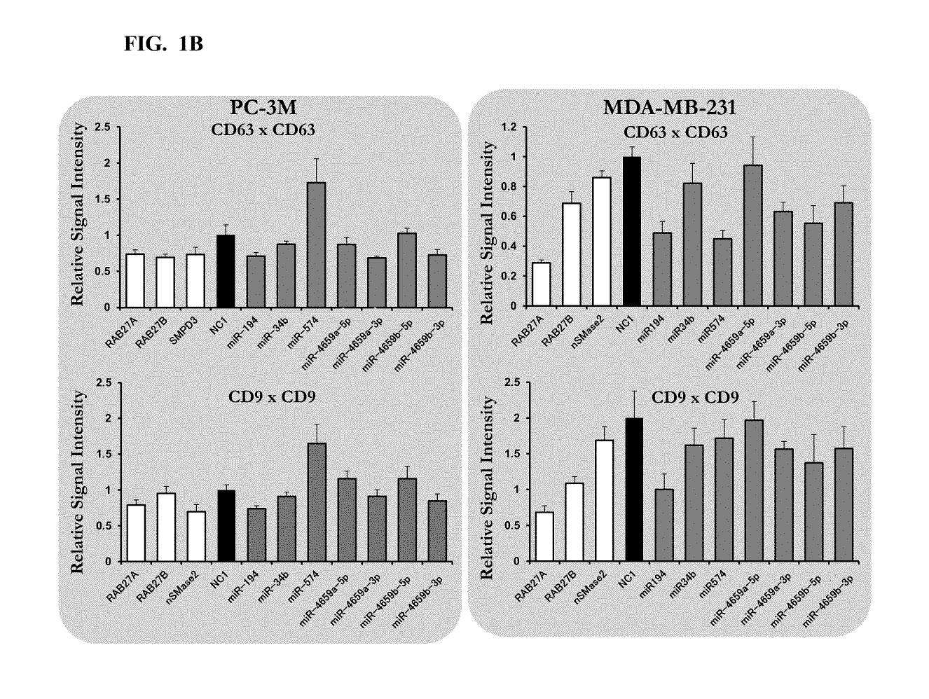

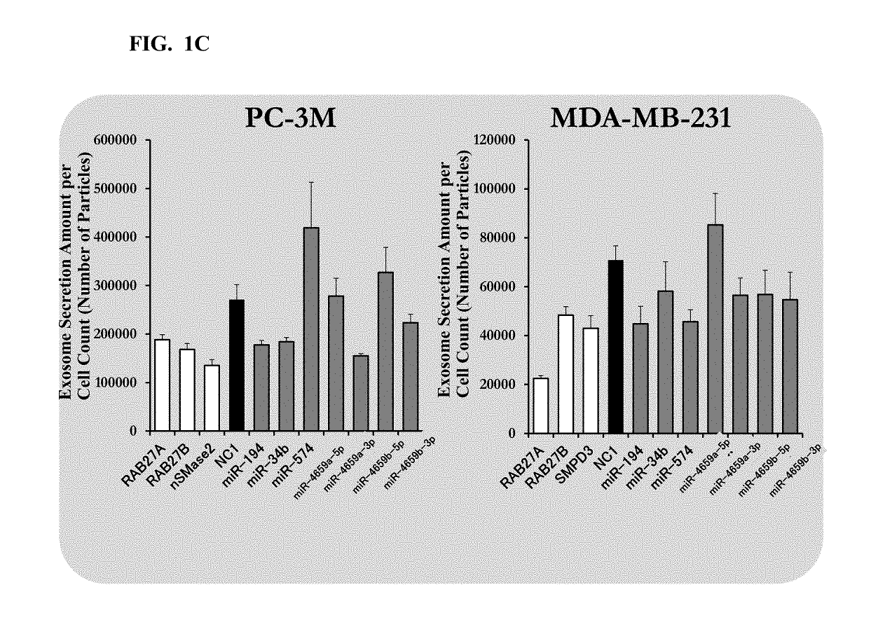

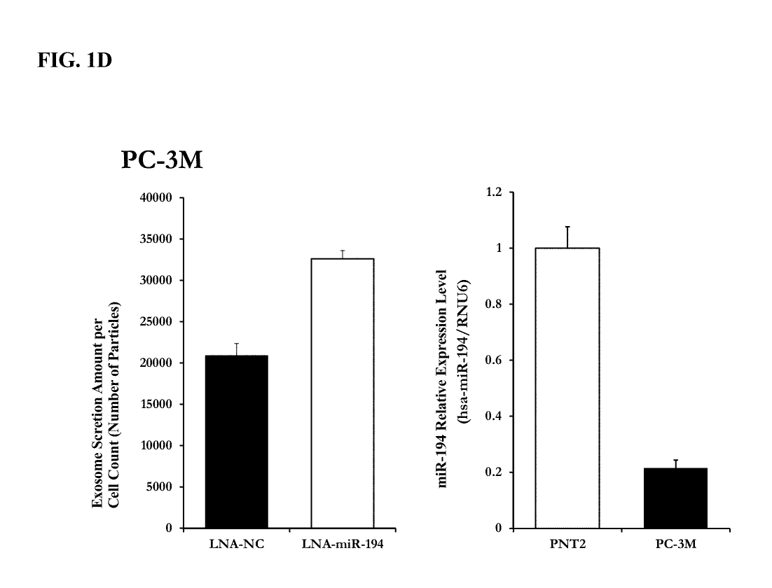

As a result of the comparison, it was found that miR-194 has suppressing action of exosome secretion (FIGS. 1A to D).

### Example 2 (Identification of Target Gene of miR-194)

The amount 1×10⁴ cells of PC-3ML cells or MDA-MB-231D3H2LN cells were seeded to each well in a serum-containing medium in a 6-well plate. Next day, the culture supernatant was exchanged with advanced RPMI (aRPMI; serum free, antibiotics free) (2 mL/well).

To a 1.5 mL tube were added 100 μL each of 2 μM miR-194 mimic (Ambion) or miR-194 LNA, and 100 μL of aRPMI. DharmaFECT1 Transfection Reagent was furnished to have a concentration of 6 μL/well, the solution was diluted with aRPMI so as to have a concentration of 10 μL/well, the diluted solution was incubated at an ordinary temperature for 5 minutes. After termination of incubation, 400 μL of the solution was added to cells in each well.

Next day, the culture supernatant was removed, and new aRPMI was added thereto in an amount of 4 mL. After additional 24 hours, the culture supernatant was collected to calculate an exosome secretion amount per cell count in the same manner as in Example 1. In addition, the transfected cells were collected and RNA extraction was carried out using miRNeasy mini kit (QIAGEN), to compare expression of each of genes by a microarray with a tip manufactured by Agilent.

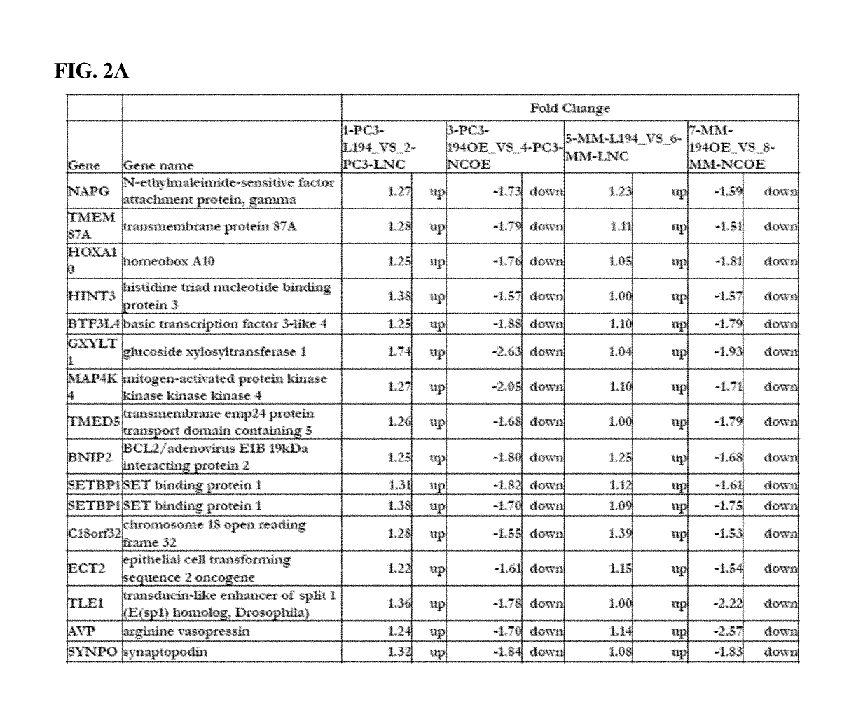

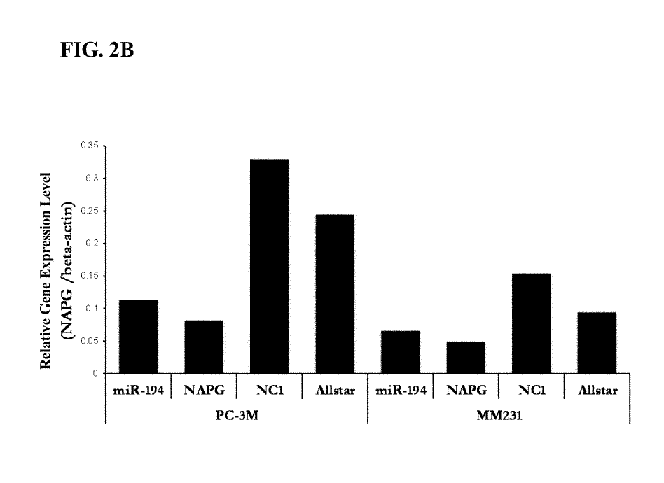

Since the gene of which expression level is lowered by administering miR-194 mimic and expression level is increased by administering miR-194 LNA is a candidate for the target gene of miR-194, genes such as NAPG were found as candidates for target genes of mir-194 (FIGS. 2A and B).

### Example 3 (Suppression of Exosome Secretion by siRNA of NAPG and TMED5)

The amount 1×10⁴ cells of PC-3ML cells or MDA-MB-231D3H2LN cells were seeded to each well in a serum-containing medium in a 96-well plate. Next day, the culture supernatant was exchanged with advanced RPMI (90 μL/well).

Each of 10 μL of siRNA (2 μM) against NAPG and TMED which were listed as candidates for target genes (see, FIG. 2A) and 10 μL of aRPMI were mixed to a separately furnished 96-well plate.

Next, DharmaFECT1 Transfection Reagent was diluted with aRPMI so as to have a concentration of 10 μL/well, the diluted solution was incubated at an ordinary temperature for 5 minutes, and aRPMI was added thereto so as to have a concentration of 90 μL/well. The solution was added thereto in an amount of 90 μL per well of a PCR plate, the mixture was incubated at an ordinary temperature for 30 minutes. After incubation at an ordinary temperature for 30 minutes, 90 μL of solution was added to each well of a 96-well plate in which PC-3ML cells or MDA-MB-231D3H2LN cells were seeded. Next day, the culture supernatant was removed and new aRPMI was added thereto in an amount of 60 μL. After additional 24 hours, 10 μL of the culture supernatant was used for quantification of an exosome secretion amount, and the remaining culture supernatant was used for quantification of the cell count. Each quantification was carried out in the same manner as in Example 1.

The exosome amount obtained was divided by the cell count to calculate an exosome secretion amount per cell count, to compare the effects of miRNAs against each target gene which was listed as a candidate.

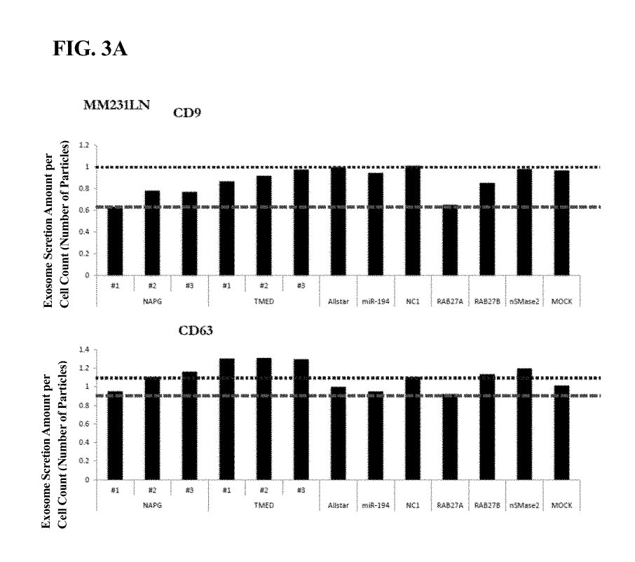

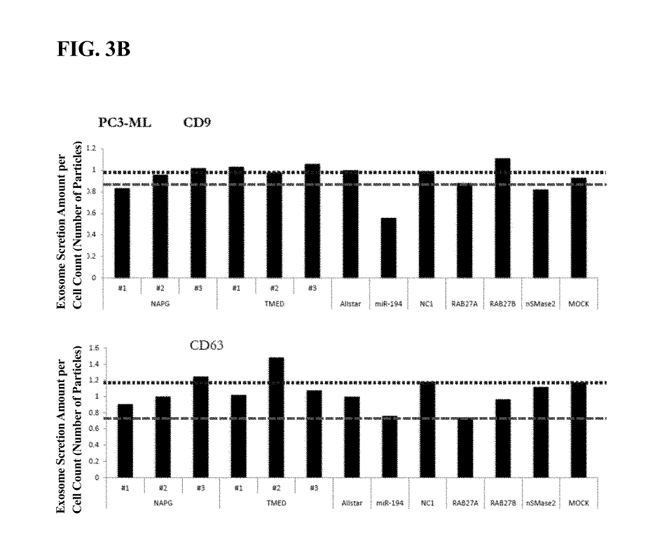

As a result of the comparison, it was found that the siRNA for NAPG has remarkable suppressing action of exosome secretion amount (FIGS. 3A and B).

### Example 4 (Measurement of Involvement of siRNA for NAPG in Exosome Secretion)

The amount 5×10⁵ cells of PC-3ML cells or MDA-MB-231D3H2LN cells were seeded to each well in a serum-containing medium in a 6-well plate. Next day, the culture supernatant was exchanged with advanced RPMI (2 mL/well).

To a 1.5 mL tube were added 100 μL of siRNA for 2 μM NAPG and 100 μL of aRPMI.

Next, DharmaFECT1 Transfection Reagent was diluted with aRPMI so as to have a concentration of 200 μL/well, the diluted solution was incubated at an ordinary temperature for 5 minutes, and the incubated solution was added to the tube containing each siRNA. After incubation at an ordinary temperature for 30 minutes, 400 μL of the mixture was added to each cell in wells.

Next day, the culture supernatant was removed, and new aRPMI was added thereto in an amount of 4 mL. After additional 24 hours, an entire amount of the culture supernatant was collected. The remaining cells were collected and the cell count was measured and counted with a hemacytometer. In addition, the collected culture supernatant was centrifuged at 2,000×g for 10 minutes, the supernatant was collected, and the collected culture supernatant was filtrated with a 0.22 μm filter. The filtrated culture supernatant was ultracentrifuged at 100,000×g for 70 min at 4° C. The supernatant was discarded, and the centrifuged product was washed with PBS at 100,000×g for 70 min. The supernatant was discarded, 200 μL of PBS was added to a ultracentrifuge tube, exosome was collected, and an exosome amount was quantified with Nanosight or microBCA assay (Pierce).

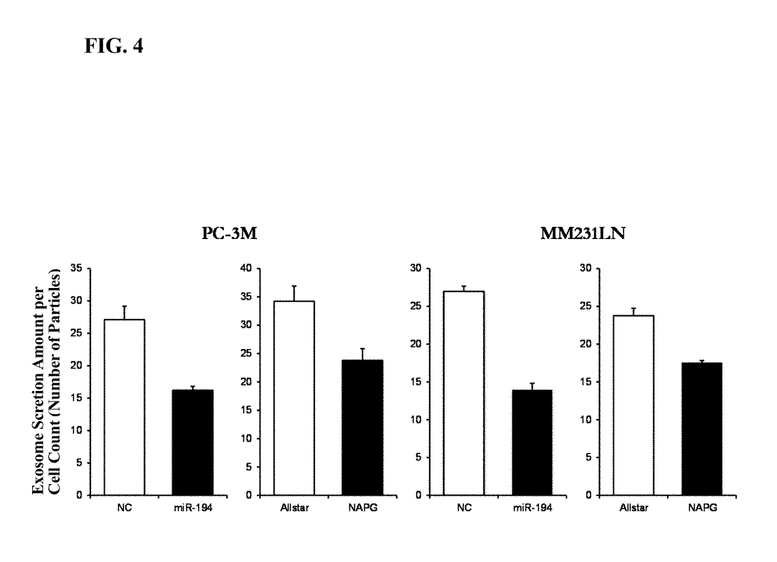

As a result, it was found that siRNA for NAPG suppresses the secretion of exosome (FIG. 4).

### Example 5 (Measurement of Involvement of siRNAs for HINT3 and GXYLT1 in Exosome Secretion)

The same analysis as in Example 3 was carried out for HINT3 and GXYLT1 genes which were found as candidates for the target genes of mir-194 in Example 2.

Concretely, the amount 1×10⁴ cells of MDA-MB-231D3H2LN cells were seeded to each well in a serum-containing medium in a 96-well plate. Next day, the culture supernatant was exchanged with advanced RPMI (90 μL/well).

To a separately furnished 96-well plate, 10 μL each of siRNAs against 2 μM HINT3 or GXYLT1 and 10 μL of aRPMI were mixed.

Next, DharmaFECT1 Transfection Reagent was diluted with aRPMI so as to have a concentration of 10 μL/well, the diluted solution was incubated at an ordinary temperature for 5 minutes, and aRPMI was added thereto so as to have a concentration of 90 μL/well. The solution was added to wells in an amount of 90 μL per well of a PCR plate, and the well was incubated at an ordinary temperature for 30 minutes. After the incubation at an ordinary temperature for 30 minutes, 90 μL each of an incubated mixture was added to each well of a 96-well plate in which MDA-MB-231D3H2LN cells were seeded. Next day, the culture supernatant was removed, and new aRPMI was added thereto in an amount of 60 μL. After additional 24 hours, 10 μL of the culture supernatant was used for quantification of an exosome secretion amount, and the remaining culture supernatant was used for quantification of the cell count. Each quantification was carried out in the same manner as in Example 1.

The obtained exosome amount was divided by the cell count to calculate an exosome secretion amount per the cell count, to compare the effects of siRNAs against each target gene which was listed as a candidate.

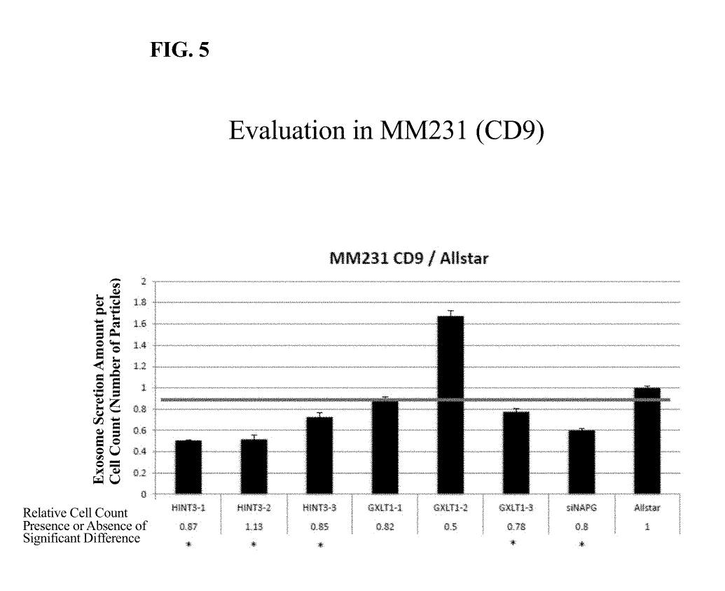

As a result of the comparison, it was found that siRNA for HINT3 and GXYLT1 have remarkable suppression action of exosome secretion (FIG. 5).

### Example 6 (Suppression of Cancer Metastasis in In Vivo Model by siRNA for NAPG)

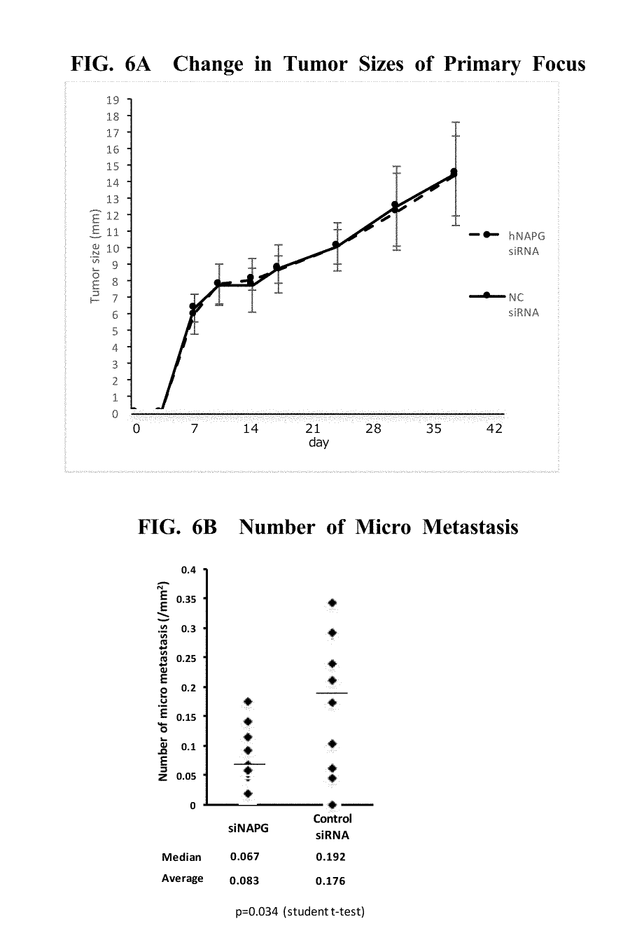

MDA-MB-231D3H2LN cells were transplanted into a mammary gland of SCID mice in an amount of 1×10⁶ cells/mouse (10 mice per group). siRNA against NAPG and siRNA which was a negative control (AllStars, QUIAGEN) were administered into a tumor with an in vivo-jetPEI (Polyplus-transfection) in an amount of 20 μg/mouse, twice per week after one week of the transplantation (for 3 weeks, 6 times administration in total). A tumor size in primary lesion was measured with a caliper once per week (FIG. 6A). After 44 days from the transplantation, the mice were anatomized, and an evaluation regarding the extent of metastasis to the lungs was made. A section of a lung tissue was prepared, and subjected to immunohistochemistry using an anti-human vimentin antibody. The number of micro metastasis which was detected as positive with the same antibody in each mouse was measured, to calculate the number of micro metastasis per 1 mm² (FIG. 6B). As a result, in mice administered with siRNA against NAPG, a difference of a tumor size of primary focus was not found, but the number of micro metastasis to a lung was significantly reduced, as compared to the mice administered with the negative control siRNA.

### INDUSTRIAL APPLICABILITY

The present invention is applicable in the field of a medicament, particularly in the fields of development and manufacture of a carcinostatic agent.

### SEQUENCE LISTING FREE TEXT

SEQ ID NO: 1 of Sequence Listing is a nucleotide sequence of mRNA of human NAPG.

SEQ ID NO: 2 of Sequence Listing is a polypeptide of human NAPG.
SEQ ID NO: 3 of Sequence Listing is a nucleotide sequence of mRNA of human HINT3.
SEQ ID NO: 4 of Sequence Listing is a polypeptide of human HINT3.
SEQ ID NO: 5 of Sequence Listing is a nucleotide sequence of mRNA of human GXLT1.
SEQ ID NO: 6 of Sequence Listing is a polypeptide of human GXLT1.

1. An exosome secretion inhibitor comprising a substance suppressing expression of NAPG, HINT3 or GXYLT1 gene, or a substance suppressing activity of NAPG, HINT3 or GXYLT1 protein.

2. The exosome secretion inhibitor according to claim 1, wherein at least one of the substance suppressing expression of NAPG, HINT3 or GXYLT1 gene, or the substance suppressing activity of NAPG, HINT3 or GXYLT1 protein is a nucleic acid.

3. The exosome secretion inhibitor according to claim 1, wherein the substance suppressing expression of the gene is one or more members selected from the group consisting of siRNA, miRNA, an antisense oligonucleotide and a ribozyme of the gene and an expression vector thereof.

4. A pharmaceutical composition comprising a substance suppressing expression of NAPG, HINT3 or GXYLT1 gene, or a substance suppressing activity of NAPG, HINT3 or GXYLT1 protein.

5. The pharmaceutical composition according to claim 4, wherein at least one of the substance suppressing expression of NAPG, HINT3 or GXYLT1 gene, or the substance suppressing activity of NAPG, HINT3 or GXYLT1 protein is a nucleic acid.

6. The pharmaceutical composition according to claim 4, wherein the substance suppressing expression of gene is one or more members selected from the group consisting of siRNA, miRNA, an antisense oligonucleotide and a ribozyme of the gene and an expression vector thereof.

7. The pharmaceutical composition according to claim 4, for use in the treatment of cancer or the suppression of cancer metastasis.

8. The pharmaceutical composition according to claim 7, wherein the cancer is colorectal cancer or breast cancer.

9. An exosome secretion inhibitor comprising miR-194.

10.-14. (canceled)
